(19) United States
(12) Patent Application Publication
(10) Pub. No.: US 2015/0335258 A1
(43) Pub. Date: Nov. 26, 2015

(54) SILICON MICROSYSTEMS FOR HIGH-THROUGHPUT ANALYSIS OF NEURAL CIRCUIT ACTIVITY, METHOD AND PROCESS FOR MAKING THE SAME

(71) Applicant: THE REGENT OF THE UNIVERSITY OF CALIFORNIA, Oakland, CA (US)

(72) Inventor: Sotiris MASMANIDIS, Pasadena, CA (US)

(21) Appl. No.: 14/648,244

(22) PCT Filed: Nov. 30, 2013
(86) PCT No.: PCT/US2013/072522

§ 371 Date: May 28, 2015

Related U.S. Application Data
(60) Provisional application No. 61/732,259, filed on Nov. 30, 2012.

(51) Int. Cl. A61B 5/04; A61B 5/00

(52) U.S. Cl. CPC A61B 5/04001; A61B 5/4064; A61B 5/6868; A61B 2562/125

(57) ABSTRACT

Provided herein are multi-electrode probe/microsystem designs that readily allow recording with multiple electrodes simultaneously, and of two spatially distinct brain regions at the same time. Also provide are methods and processes for manufacturing the probes.

### CROSS-REFERENCE TO RELATED APPLICATION

This application claims priority to U.S. provisional patent application No. 61/732,259 filed on Nov. 30, 2012 and entitled “SILICON MICROSYSTEMS FOR HIGH-THROUGHPUT ANALYSIS OF NEURAL CIRCUIT ACTIVITY, METHOD AND PROCESS FOR MAKING THE SAME,” which is hereby incorporated by reference herein in its entirety.

### FIELD OF THE INVENTION

The present invention relates to a novel process for manufacturing microsystems, which utilizes photolithography, etching and any other applicable methods and techniques. More specifically, the present invention relates to silicon microsystems containing implantable electrodes for measuring electrical signaling in the brain at the resolution of multiple single neurons, their designs, methods and processes for making the same.

### BACKGROUND OF THE INVENTION

Large-scale studies of gene expression in the brain, such as the Allen Brain Atlas and the GENS AT project, have revolutionized the ability for identifying molecular markers for subpopulations of neurons in different anatomical regions. At the same time, the development of genetically encoded activators and silencers of neuronal activity have provided enormous new opportunities for studying the role of brain circuitry in behavior. Large-scale network activity is an important link between the gain or loss of function of genetically defined neuronal subpopulations, and specific behaviors. However, monitoring network phenomena has been a major technological obstacle in systems neuroscience. By surmounting this problem a putative causal relation can be established between the activation/silencing of a subset of genetically defined neurons, the evoked pattern of action potentials across several anatomical locations, and a specific behavior or cognitive function. Efforts to scale up the capabilities of electrophysiological measurements with inexpensive, disposable devices are therefore important and timely, thanks to the rising use of genetic brain circuitry functional manipulation techniques. Furthermore, efforts to improve the therapeutic value of neuropsychopharmaceuticals must increasingly rely on an understanding of the underlying brain circuitry—such understanding remains incomplete.

Currently available commercial neural probes, typically based on silicon microfabrication processes are expensive ($100's to $1,000's per single chip) and do not meet the increasingly ambitious needs of the research community. It is clear that the next revolution in neural probe technology will arise by tackling both problems of cost (through more efficient production methods) and function (through enhanced recording performance of the devices). The probe design described here addresses the second need of enhanced recording performance.

Implantable multi-electrode silicon probes (Blanche et al., 2005; Campbell et al., 1991; Drake et al., 1988; Najafi et al., 1985; Norlin et al., 2002; Wise 2007) has led to great advances in large scale recording with high {i.e., single-cell) resolution. However, current tools fall short of providing a densely populated ‘activity map,’ which may offer a better understanding of the circuitry of cell assemblies in the brain. Such maps are poised to offer new unprecedented insights into the workings of the mind in health and disease, and also provide valuable information on the effect of pharmaceuticals in the treatment of a broad spectrum of neurological disorders such as Parkinson's disease, depression, and OCD. This invention addresses the shortcomings of current micromachined neural probes, by relying on a novel approach to mass-producing silicon probes, offering consumers better products than competing devices in terms of their function (more recording electrodes are packed onto a single device translating to more useful information gleaned about the brain), device size (our devices utilize smaller feature size than competing devices, thereby reducing tissue damage), and scale of production (lower cost translates to disposable devices, thereby opening the market to more users).

Information on existing design of multi-electrode silicon probes can be found in, for example, J. Du et al., 2011, Multiplexed, “High-density electrophysiology with nanofabricated neural probes,” PloS ONE e26204; and U.S. Pat. No. 8,355,768 to Masmanidis et al. and entitled “Micromachined neural probes;” each of which is hereby incorporated by reference herein in its entirety. Commercial products are available through Neuronexus Technologies (Ann Harbor, Mich.).

There is an increasing demand in neuroscience for large scale recording of neuronal activity (Buzsaki 2004). Techniques such as electroencephalography (EEG) and functional magnetic resonance imaging (fMRI) provide coarse grained views on synchronized activity, but they do not afford much insight into the brain's circuitry at the level of single neurons.

What is needed in the art are better designed probes and more efficient methods/processes for making the probes.

### SUMMARY OF THE INVENTION

Provided herein is a fabrication process that is less complex, driving down the cost of production and making the prospect of disposable devices feasible for the first time. Also provided herein are the critical device dimensions offered by this manufacturing process are about 5-fold less than competing fabrication methods, allowing the development of narrower, hence less invasive neural interfaces.

Provided herein are probes of multi-electrode designs that readily allow recording with multiple electrodes simultaneously, and of two spatially distinct brain regions at the same time.

In one aspect, a process for manufacturing a device comprising a plurality of electrical elements is provided. The process comprises the steps of: applying a first module in direct contact with a first electrical element layer of a pre-treated substrate, wherein the first electrical element layer is selected from the group consisting of a metal layer, an insulating layer, a semiconductor layer, a layer of photoresist, and a combination thereof, and wherein the first module comprises, on a surface that forms direct contact with the first electrical element layer, a first template design; and printing a first pattern on the first electrical element layer, wherein the first pattern is formed by a first material having a photo sensitivity.

In some embodiments, the first electrical element layer has a first photo sensitivity that is different from the photo sensitivity of the first material. In any of the preceding embodiments the first material is a photoresist and the first pattern is exposed to ultraviolet radiation. In any of the preceding embodiments, the process further comprises a step of creating a first plurality of electrical elements of the device using one or more methods selected from the group consisting of ultraviolet photolithography, etching, deep reactive ion etching, ion milling, grinding, polishing, and combinations thereof.

In any of the preceding embodiments, the process further comprises a step of creating the first template design on the first module, wherein the first template design corresponding to an arrangement of a first plurality of electrical elements of the device, and wherein at least one electrical element of the first plurality is selected from the group consisting of an electrode, an electrical lead, an insulator, a contact pad, a semiconductor, and a combination thereof.

In any of the preceding embodiments, the process further comprises a step of depositing a second electrical element layer above the first electrical element layer, wherein the second electrical element layer is selected from the group consisting of a metal layer, an insulating layer, a semiconductor layer, a layer of photoresist, and a combination thereof.

In any of the preceding embodiments, the second electrical element layer is different from the first electrical element layer.

In any of the preceding embodiments, the process further comprises a step of applying a second module in direct contact with the second electrical element layer, wherein the second module comprises, on a surface that forms direct contact with the second electrical element layer, a second template design; and printing a second pattern on the second electrical element layer, wherein the second pattern is formed by a second material having a photo sensitivity.

In any of the preceding embodiments, the second electrical element layer has a second photo sensitivity that is different from the photo sensitivity of the material forming the second pattern. In any of the preceding embodiments, the second material is a photoresist and the second pattern is exposed to ultraviolet radiation.

In any of the preceding embodiments, the process further comprises a step of creating a second plurality of electrical elements of the device using one or more methods selected from the group consisting of ultraviolet photolithography, etching, deep reactive ion etching, ion milling, grinding, polishing, and combinations thereof.

In any of the preceding embodiments, the process further comprises a step of creating the second template design on the second module, wherein the second template design corresponding to an arrangement of a second plurality of electrical elements of the device, and wherein at least one electrical element of the second plurality is selected from the group consisting of an electrode, an electrical lead, an insulator, a contact pad, a semiconductor, and a combination thereof.

In any of the preceding embodiments, the process further comprises a step of depositing a third electrical element layer above the second electrical element layer, wherein the third electrical element layer is selected from the group consisting of a metal layer, an insulating layer, a semiconductor layer, a layer of photoresist, and a combination thereof.

In any of the preceding embodiments, the third electrical element layer is different from the second electrical element layer.

In any of the preceding embodiments, the process further comprises a step of applying a third module in direct contact with the third electrical element layer, wherein the third module comprises, on a surface that forms direct contact with the third electrical element layer, a third template design; and printing a third pattern on the third electrical element layer, wherein the third pattern is formed by a third material having a photo sensitivity.

In any of the preceding embodiments, the third electrical element layer has a third photo sensitivity that is different from the photo sensitivity of the third material. In any of the preceding embodiments, the third material is a photoresist and the third pattern is exposed to ultraviolet radiation. In any of the preceding embodiments, the process further comprises a step of creating a second plurality of electrical elements of the device using one or more methods selected from the group consisting of ultraviolet photolithography, etching, deep reactive ion etching, ion milling, grinding, polishing, and combinations thereof.

In any of the preceding embodiments, the process further comprises a step of creating the third template design on the third module, wherein the third template design corresponding to an arrangement of a third plurality of electrical elements of the device, and wherein at least one electrical element of the third plurality is selected from the group consisting of an electrode, an electrical lead, an insulator, a contact pad, a semiconductor, and a combination thereof.

In any of the preceding embodiments, the pre-treated substrate further comprises: a base material selected from the group consisting of silicon, quartz, silicon oxide, sapphire, gallium arsenide, magnesium oxide, zinc oxide, and silicon carbide. In any of the preceding embodiments, the base material further comprises a buried oxide (BOX) layer.

In any of the preceding embodiments, the first material is a photoresist selected from the group consisting of a negative photoresist and a positive photoresist. In any of the preceding embodiments, the second material is a photoresist selected from the group consisting of a negative photoresist and a positive photoresist. In any of the preceding embodiments, the third material is a photoresist selected from the group consisting of a negative photoresist and a positive photoresist.

In one aspect, also provided herein is a device comprising a plurality of electrical elements manufactured according to a combination of any of the methods described herein.

In some embodiments, the device a first implantable shaft; a first plurality of electrodes disposed on the first implantable shaft; a second implantable shaft; a second plurality of electrodes disposed on the second implantable shaft; a base to which the first implantable shaft and the second implantable shaft are attached and separated by a first distance; and at least one contact pad on the base to which one electrode from the first plurality of electrodes and one electrode from the second plurality of electrodes are connected.

In one aspect, also provided herein is a neural probe. The probe comprises a first implantable shaft; a first plurality of electrodes disposed on the first implantable shaft; a second implantable shaft; a second plurality of electrodes disposed on the second implantable shaft; a base to which the first implantable shaft and the second implantable shaft are attached and separated by a distance; and at least one contact pad on the base to which one electrode from the first plurality of electrodes and one electrode from the second plurality of electrodes are connected.

In any of the preceding embodiments, the distance separating the first implantable shaft and the second implantable shaft is adjustable. In any of the preceding embodiments, electrodes in the first plurality of electrodes or second plurality of electrodes are capable of measuring electrical signals from multiple single neurons. In any of the preceding embodiments, each electrode in the first plurality of electrodes and second plurality of electrodes is connected to a contact pad.

In any of the preceding embodiments, the neural probe further comprises a first plurality of contact pads, wherein each contact pad of the first plurality of contact pads is connected to two or more electrodes, and wherein at least two electrodes are disposed on different implantable shafts.

In any of the preceding embodiments, each contact pad of the first plurality of contact pads is connected to two electrodes that are disposed on different implantable shafts. In any of the preceding embodiments, the neural probe further comprises a second plurality of contact pads, wherein each contact pad of the second plurality of contact pads is connected to two or more electrodes, and wherein at least two electrodes are disposed on different implantable shafts. In any of the preceding embodiments, each contact pad of the second plurality of contact pads is connected to two electrodes that are disposed on different implantable shafts.

In any of the preceding embodiments, a first Application Specific Integrated Circuits (ASIC) is in contact with contact pads in the first plurality of contact pads. In any of the preceding embodiments, a second Application Specific Integrated Circuits (ASIC) is in contact with contact pads in the second plurality of contact pads.

### BRIEF DESCRIPTION OF THE DRAWINGS

Those of skill in the art will understand that the drawings, described below, are for illustrative purposes only. The drawings are not intended to limit the scope of the present teachings in any way.

FIGS. 1A and 1B depicts exemplary embodiments of conventional photolithography.

FIGS. 2A-2I depict an exemplary process in accordance with the present invention.

FIGS. 3A and 3B depict exemplary processes in accordance with the present invention.

FIG. 4 depicts an exemplary design for a neural probe for targeting multiple brain structures. Each contact pad is connected to two microelectrodes; each one on a separate shaft. The microelectrodes on the left hand side shafts are spaced by a distance A, and the microelectrodes on the right hand side shafts are spaced by a distance B. To use the “A” spacing the user would physically break the two shafts on the right by mechanical force. The shafts would break near the specified break point. Conversely, to use the “B” spacing the user would physically break the two shafts on the left.

FIG. 5 depicts an exemplary approach to directly connecting silicon probes to multiple Application-Specific Integrated Circuits (ASICs). The number of electrical channels per probe can be effectively doubled by duplicating the contacts around the midline of the probe, and bonding one ASIC per side.

### DETAILED DESCRIPTION OF THE INVENTION

The terms “microsystem” and “probe” will be construed broadly and used interchangeably.

The term “electrical elements” refers to any structural or functional component that can be used to form an electrical arrangement such as an integrated circuit. As used herein, an electrical element itself does not have to conduct electricity. Exemplary electrical elements include but are not limited to an electrode, an electrical lead, a contact pad, an insulator, a semiconductor element (e.g., diodes, transistors and etc.), or a combination thereof.

### Manufacturing Process

In one aspect, devices with one or more electrical elements, such as an electrode, an electrical lead, a contact pad, an insulator, a semiconductor element, and a combination thereof, are manufactured by a novel manufacturing process that utilizes photolithography, etching and any other applicable methods and techniques. In particular, the methods and processes described herein eliminate the needs of using photomasks, thereby improving efficiency and accuracy, and reducing the costs.

### Conventional Photolithography

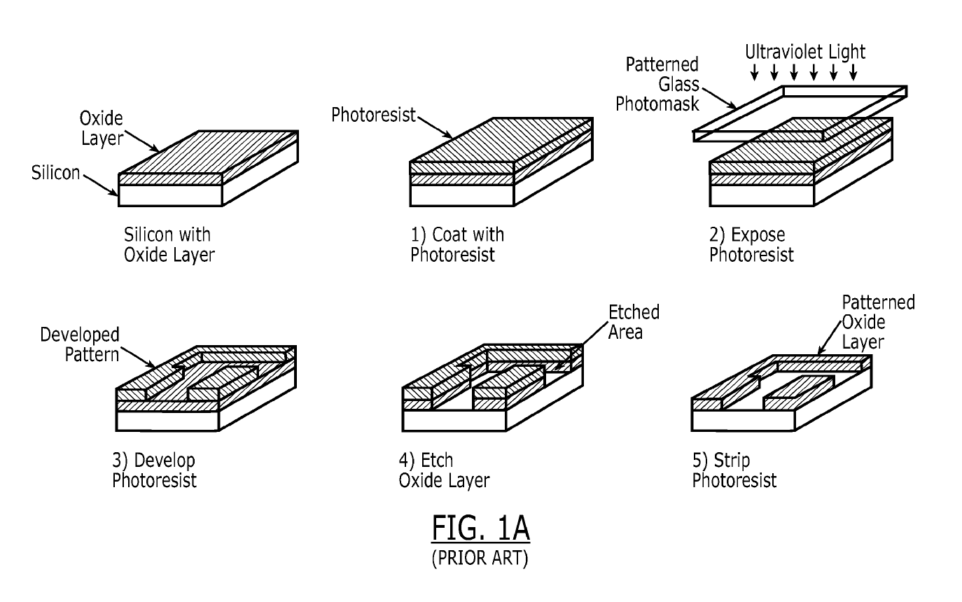

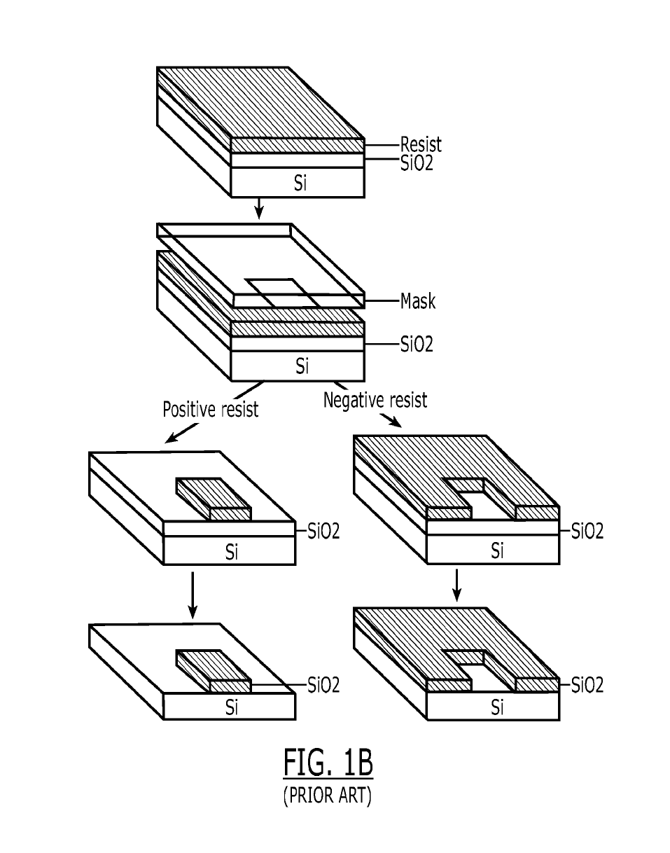

In conventional photolithography techniques, photomasks are used in conjunction with photoresist (positive or negative) to produce electrical elements comprising electrodes, electrical leads, insulators, semiconductor elements, and contact pads by photolithography. Exemplary conventional photolithography processes are depicted in FIGS. 1A and 1B.

In general, a mask or “photomask” is a glass plate with a patterned emulsion of metal film on one side. The pattern generally corresponds to arrangements of electrical elements such as a design for an integrated circuit. The mask is aligned with a wafer substrate, so that the pattern can be transferred onto the wafer surface. Each mask after the first one must be aligned to the previous pattern. Once the mask is aligned with the pattern on the wafer's surface, the photoresist is exposed through the pattern on the mask with a high intensity ultraviolet (UV) light.

Referring to FIG. 1A, a silicon substrate coated with an oxide layer is used as the starting material. In step 1), a layer of photoresist is deposited on the oxide layer. In step 2), a patterned glass use aligned with the substrate through which the layer of photoresist is exposed to ultraviolet radiation. In step 3), the photoresist layer is developed and the photoresist is the area exposed to ultraviolet radiation is dissolved in developer solution, leaving areas that are inversely correlated with the transparent pattern on the photomask. In step 4), areas in the oxide layer that are not protected by developed photoresist are then removed by methods such as etching (e.g., using hydrofluoric acid or dry etching). In step 5), the developed photoresist is stripped off, revealing the patterned oxide layer.

Two types of UV photoresist: positive and negative, can be used for photolithography. For positive resists, the resist is exposed with UV light wherever the underlying material is to be removed. In these resists, exposure to the UV light changes the chemical structure of the resist so that it becomes more soluble in the developer. The exposed resist is then washed away by the developer solution, leaving windows of the bare underlying material. The mask, therefore, contains an exact copy of the pattern which is to remain on the wafer. Negative resists behave in just the opposite manner. Exposure to the UV light causes the negative resist to become polymerized, and more difficult to dissolve. Therefore, the negative resist remains on the surface wherever it is exposed, and the developer solution removes only the unexposed portions. Masks used for negative photoresists, therefore, contain the inverse (or photographic “negative”) of the pattern to be transferred. FIG. 1B illustrates the different patterns generated from the use of positive and negative resist. In the process depicted in FIG. 1A, positive photoresist is used to remove the areas exposed to UV radiation.

One of the most important steps in the conventional photolithography process is mask alignment. There are three primary exposure methods: contact, proximity, and projection. The pattern embedded in photomask contains electrical elements of very small size, for example, down to sub-micron level. Constructive or destructive interferences of the UV light occur, and the pattern created on the wafer is often different from the intended design, even when the effects of interferences are taken into consideration. As a result, photomask-based conventional photolithography can be wasteful, costly, and inaccurate.

### Novel Photolithography Cycle

In the novel photolithography manufacturing process described hereinbelow, the need for a photomask has been eliminated. Instead, a stamp-like module is used. The module bears a template design corresponding to the features of any desired electrical elements (e.g., electrodes, electrical leads, insulators, semiconductors and contact pads and etc.).

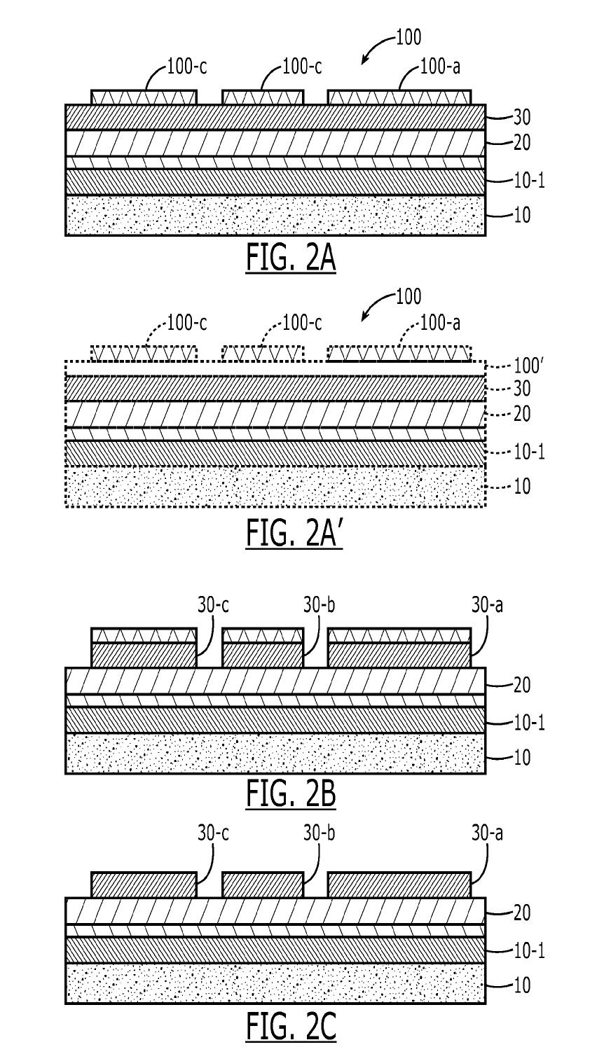

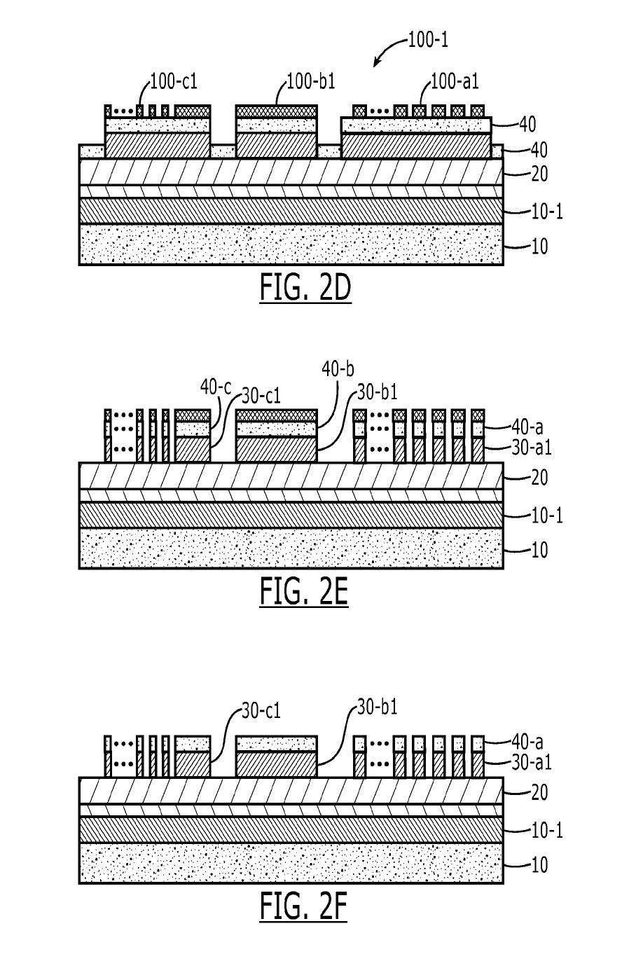

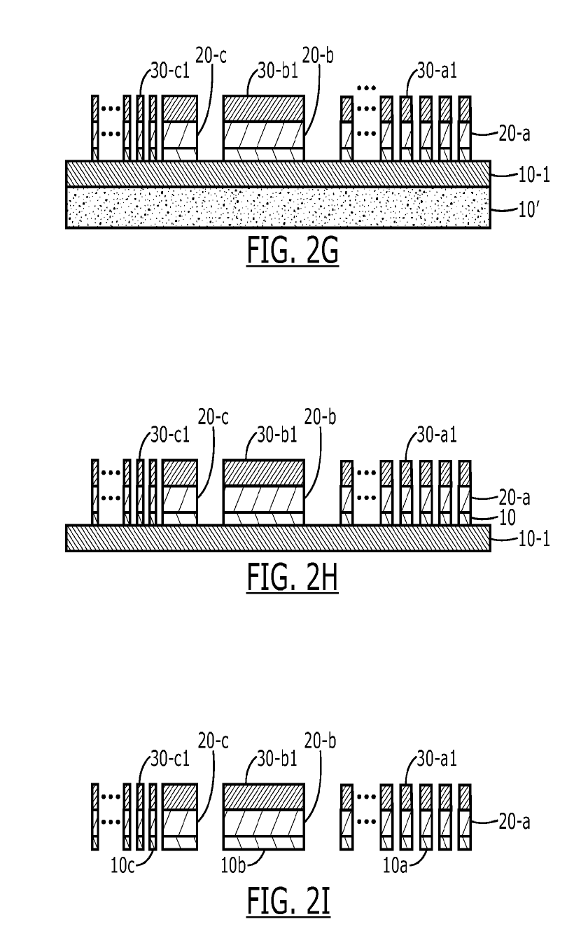

FIG. 2 depicts an exemplary manufacturing process. It will be understood that alternative selections of substrate are alternative arrangements of layers of the substrates and layers deposited on either or both sides of the substrate are possible. The sequence depicted in FIG. 2 is only provided as an illustration and should not in any way limit the scope of the invention. It will be understood that FIG. 2A through FIG. 2I depict only a particular section of a layered structure. Regions or parts that are shown as discrete or separate units in the cross-sectional view can be connected.

Referring to FIG. 2A, a substrate 10 serves the structural support. In some embodiments, as shown in FIG. 2, substrate 10 includes a buried oxide (BOX) layer 10-1. In some embodiments, no BOX layer is used. Any suitable material can be used as substrate 10. In some embodiments, substrate 10 is a semiconductor material such as silicon, silicon oxide such as quartz, sapphire, gallium arsenide, magnesium oxide, zinc oxide, and silicon carbide. In some embodiments, a non-semiconductor material (e.g., glass, organic polymer) is used as substrate 10, including but not limited to parylene, diamond, plastic, ceramic. For the purpose of this invention, substrate 10 can be of any applicable size, shape and dimension; for example, circular, square or rectangle. In some embodiments, the largest dimension of the substrate can be 500 mm or smaller, 450 mm or smaller, 400 mm or smaller, 350 mm or smaller, 300 mm or smaller, 250 mm or smaller, 200 mm or smaller, 150 mm or smaller, 100 mm or smaller, 50 mm or smaller, 25 mm or smaller. In some embodiments, the largest dimension of the substrate can be larger than 500 mm. In some embodiments, substrate 10 is a square or circular silicon-on-oxide (SOI) wafer of 150 mm with a BOX layer 10-1. In some embodiments, substrate 10 is a square or circular silicon-on-oxide (SOI) wafer of 150 mm without a BOX layer 10-1. In some embodiments, substrate 10 is a square silicon-on-oxide (SOI) wafer of 200 mm or 100 mm with a BOX layer 10-1. In some embodiments, substrate 10 is a circular silicon-on-oxide (SOI) wafer of 200 mm or 100 mm with a BOX layer 10-1. In some embodiments, substrate 10 has a thickness of, for example, about 500 μm or less, about 450 μm or less, about 400 μm or less, about 350 μm or less, about 300 μm or less, about 250 μm or less, about 200 μm or less, about 150 μm or less, about 100 μm or less, about 75 μm or less, about 50 μm or less, about 25 μm or less, or about 15 μm or less, about 10 μm or less, or about 5 μm or less. In one embodiment, substrate 10 has a thickness of between about 25 and about 50 nm.

In some embodiments, the substrate is be first thermally oxidized to yield a oxide layer (e.g., layer 20) on one or both sides. In some embodiments, an insulator layer 20 is deposited on the surface of substrate 10, as depicted in FIG. 2A. Insulator layer 20 can be formed by any applicable insulating material, including but not limited to silicon nitride, silicon oxide, polyimibe, parylene (parylene C) and other polymeric insulating material. Insulating layer 20 can have thickness of a about 50 μm or less, about 25 μm or less, or about 15 μm or less, about 10 μm or less, or about 5 μm or less.

A metal layer 30 is blanket-deposited over insulator layer 20. In some embodiments, metal layer 30 is formed by a suitable metal such as chromium, platinum, gold, iridium, titanium, cobalt, copper, molybdenum, or any combination thereof. In some embodiments, the metal layer comprises a 30 Å of chromium adhesion layer followed by 1500 Å of gold. In some embodiments, a Co—Cr—Mo alloy can be used. Besides their use as electrical conduit, certain metals also function as structural support. In some embodiments, a layer of copper (Cu) buffer is deposited, either directed onto substrate 10 or over insulator layer 20, before metal layer 30 is deposited. In such embodiments, dissolution of the Cu buffer releases electrical elements once they are released.

Using, for example, a stepper machine, a module is used to print a pattern 100 (e.g., formed by elements 100-a, 100-b and 100-c and etc.) directly on metal layer 30. The module is a stamp-like apparatus. For example, 100-a can correspond to a region containing multiple contact pads; 100-b can correspond to a region containing multiple electrical leads; and 100-c can correspond to a region containing multiple microelectrodes and electrical leads.

On one surface of the module, there is a pre-formed template design that corresponds to the pattern printed on metal layer 30 or any other suitable layer. The template design (and the printed pattern) corresponds to an arrangement of electrical elements. In some embodiments, the template design contains carved out features of various sizes and shapes, each corresponding to an electrical element in an arrangement of electrical elements. This way, a pattern printed is the same as the arrangement of electrical elements (i.e., it is a positive copy of the arrangement). In some embodiments, the template design contains carved out features of various sizes and shapes, each corresponding to a feature complementary to an electrical element in an arrangement of electrical elements. This way, a pattern printed is the opposite of the arrangement of electrical elements (i.e., it is a negative copy of the arrangement). In some embodiments, the template design (and the printed pattern) includes information of all electrical elements of a particular device such as a probe, or an implant. In some embodiments, information of all electrical elements of a device is implemented in multiple template designs (and multiple modules) in a iterative process. The module uses a UV photoresist as “ink” or “paint.” As such, the pattern printed on metal layer 30 is formed of the UV photoresist. Upon exposure to UV radiation, the UV photoresist is resistant to developer solution. As such, areas covered by the printed pattern of UV photoresist is protected from further processing such as etching by hydrofluoric acid, dry etching, or ion milling, as depicted in FIG. 2B. The photoresist can then be removed to reveal the metal layer underneath, which now bear a pattern corresponding to the template design in the module. For example, 30-a is a region on metal layer 30 that contains multiple contact pads; 30-b is a region on metal layer 30 that contains multiple electrical leads; and 30-c is a region on metal layer 30 that contains multiple microelectrodes and electrical leads. For ease of illustration, these regions are shown in the FIG. 2 as discrete and separated regions. However, one of skill in the art would understand that regions 30-a, 30-b and 30-c can either collectively form integrated device or exist as discrete components of one or more devices.

In alternative embodiments (e.g., FIG. 2A′), a photoresist layer 100′ is applied over metal layer 30 before printed pattern 100 is formed on the photoresist layer 100′. In some embodiments, photoresist used as ink or paint in forming the printed pattern has a photo sensitivity that is opposite to the photo sensitivity of photoresist layer 100′. In these embodiments, by selecting positive or negative photoresist as ink or paint, it is possible to create an arrangement of electrical elements that is identical or complements the arrangement of the template design in the stamp-like module.

In other alternative embodiments, stamping with the module on photoresist layer 100′ removes the photoresist in areas corresponding to the template design on the module, thereby removing the photo sensitivity from these areas.

In still alternative embodiments, the layer that directly forms contact with the stamping module is an insulator layer, an insulator layer covered by a photoresist layer, a semiconductor layer, or a semiconductor layer covered by a photoresist layer.

In these alternative embodiments, layers arranged in these embodiments can then go through similar etching or photoresist removal process as depicted in FIGS. 2B and 2C. In such embodiments, the stepper machine can be programed to stamp a desired pattern repeatedly over photoresist layer 100′.

The stepper machine can be programed to stamp a module containing a desired pattern repeatedly over metal layer 30 or photoresist layer 100′ or any other suitable layer. In some embodiments, the module refills on photoresist between stamp steps. In some embodiments, the module can contain a chamber filled with photoresist and the chamber opening is connected to the template design. For example, photoresist is suspended by vacuum in the chamber. A controlled pressure is applied to the photoresist chamber when the module surface directly contacts the surface of metal layer 30 such that photoresist fills the carved out features in template design (which corresponds to an arrangement of electrical elements; e.g., as a positive or negative). A computer system can be used to control the pressure on the photoresist chamber and coordinate with the speed at which a pattern is printed. In these embodiments, a module can repeatedly stamp the entire substrate without refilling photoresist. In some embodiments, the module can be a tubular structure (a hollow cylinder) bearing a template design on its outer surface except the circular ends. In such embodiments, the template design can be rolled onto a surface of any layer to create a continuous printed pattern. In such embodiments, the hollow center of the tubular module can be used as a photoresist chamber and photoresist can be released on the surface of any layer by gravity. This type of roller printing techniques known in new paper printing industry can be applied here.

Referring to FIG. 2D, a new layer 40 (e.g., a layer of insulator, photoresist or a new metal layer) is deposited over regions 30-a, 30-b, 30-c and etc. A new pattern 100-1 (formed by 100-a1, 100-b1, 100-c1 and etc.) is printed onto layer 40. For example, 100-a1 can correspond to multiple contact pads; 100-b1 can correspond to multiple electrical leads; and 100-c1 can correspond to multiple microelectrodes and electrical leads.

Using processes similar to those used in connection with FIG. 2B, layer 40 and layer 30 are either etched or milled according to the printed pattern. The result is more defined features in metal layer 30 and layer 40; for example, 30-a1 includes multiple contact pads; 30-b1 includes multiple electrical leads; and 30-c1 includes multiple microelectrodes and electrical leads. Thus, more refined electrical elements are defined by using two template designs in two photolithography cycles in a sequential fashion.

One of skill in the art would understand that it is possible to create the arrangement represented by 30-a1, 30-b1, 30-c1 and etc. in a single photolithography cycle, for example, by using a module with a more complex template design that includes the fine features of electrical elements in the arrangement.

One of skill in the art would also understand that it is possible to create more complex and multiple dimensional devices by employing multiple photolithography cycles and a multiple layered structure. For example, the top photoresist layer from FIG. 2E can be removed (e.g., FIG. 2F) and a new layer can be deposited on layer 40. Like layer 40, the new layer can be a new metal layer, a layer of insulator, a photoresist layer, or a combination thereof. As noted above, regions 30-a, 30-b, and 30-c. can either collectively form integrated device or exist as discrete components of one or more devices. The same applies to elements 30-a1, 30-b1, and 30-c1. The same also applies to elements 30-a1, 30-b1, and 30-c1 in any combination with elements 20-a, 20-b, and 20-c.

In some embodiments, a third lithography cycle can be applied and dry etching can be used to etch through the insulator layer 20 and the top of the substrate layer to reveal BOX layer 10-1 (e.g., FIG. 2G).

### Post-Printing and Post-Production Processing

After the features of an arrangement of all electrical elements of a device are printed and created by the processes and methods described herein, the substrate (e.g., the SOI wafer) bearing the electrical elements undergo post-printing processing. For example, the SOI wafer can be mounted upside down (e.g., via surface 10′ as depicted in FIG. 2G) onto a carrier using a water soluble adhesive (e.g., Crystalbond 555 wax). Any material that can be used as a substrate can be used as a carrier, including but not limited to silicon wafer, metal oxide (magnesium oxide, Zinc oxide and etc.), gallium nitride, silicon nitride,

In some embodiments, deep reactive ion etching (DRIE) is used to etch the substrate layer from surface 10′ until the BOX layer is reached. In some embodiments, other dry etching method can be used to remove the remaining BOX layer. In some embodiments, DRIE is used to remove the substrate layer from surface 10′ and the entire BOX layer.

Optionally, in some embodiments and before DRIE or other dry etching method is used, the substrate can be thinned down from surface 10′ using a wafer grinding or polishing tool until there is only a limited thickness remains in the substrate (e.g., about 50 to 150 microns).

After all substrate material is removed, the devices can be released by pouring hot water over the carrier or immersing the carrier in hot water. The temperature of the hot water can be between a temperature around the melting temperature of the water-soluble adhesive to about 100° C. In some embodiments, the temperature of the hot water is between 60° C. to 100° C.

After the individual devices are released from the carrier, they are fished out with tweezers or a vacuum wand. In some embodiments, the cleaning individual devices in deionized water, acetone, and ethanol or a subset of these solvents. In some embodiments, the devices are dried before being packaged for storage or user.

In some embodiments, it is possible to accelerate the cleaning process by placing the devices in an ultrasonic bath for a few seconds to a few minutes while immersed in one of the above solvents.

In some embodiments, the devices are operated by assembling them onto a printed circuit board containing a connector, passive and active electronic components, or a subset of those components, and directly implanting them in the brain of a live animal (such as, mouse, rat, bird, human) and recording the ensuing neuronal activity.

### Overall Process Description

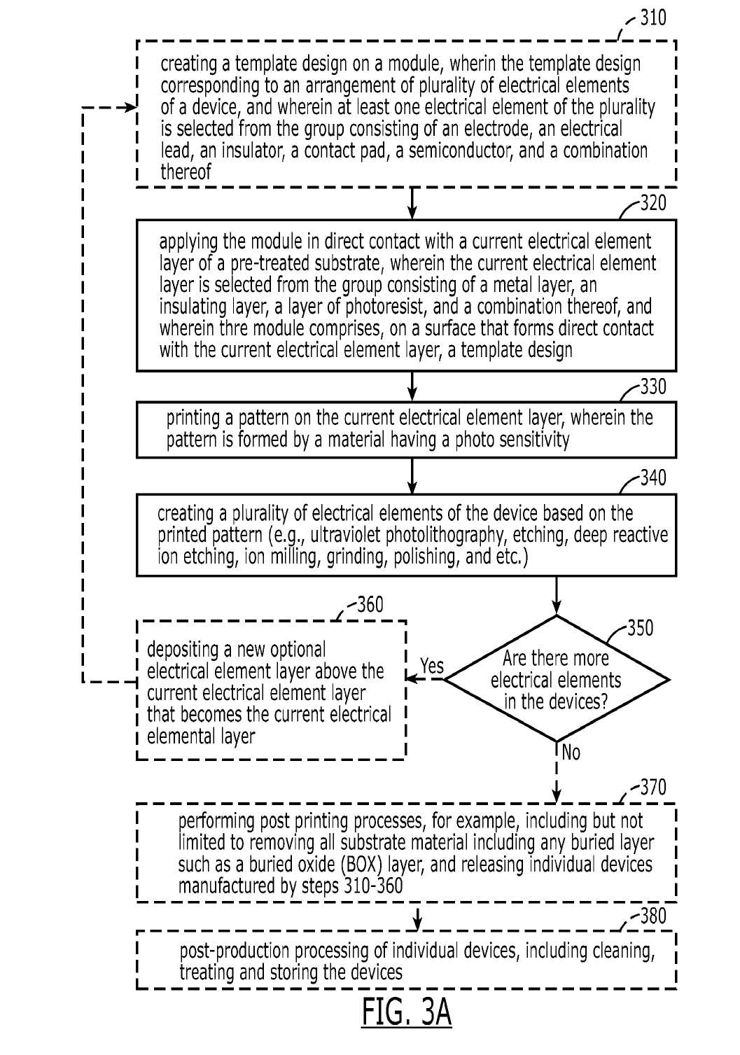

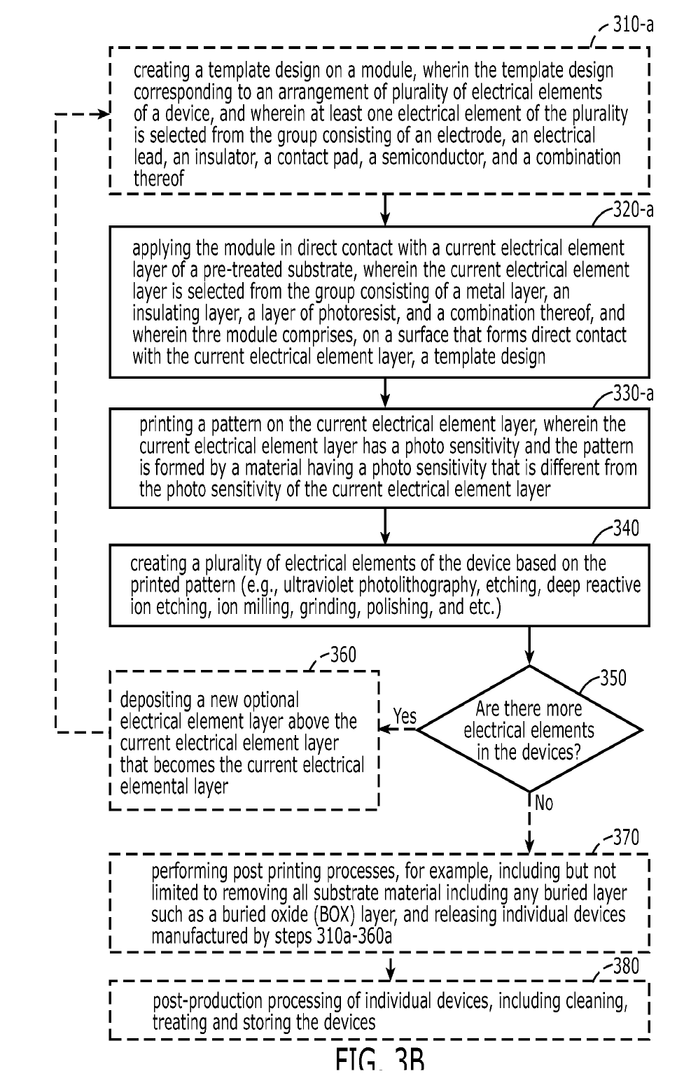

Exemplary processes for manufacturing electrical devices such as neural probes are illustrated in flow charts in FIGS. 3A and 3B. Optional steps in the processes depicted in dashed box. Also, each boxed step in the flow chart can include multiple processing or manufacturing steps.

At step 310, a template design is created on a module. The template design corresponds to an arrangement of a plurality of electrical elements of an electrical device. At least one electrical element of the plurality of electrical elements is selected from the group consisting of an electrode, an electrical lead, an insulator, a contact pad, a semiconductor, and a combination thereof. The module can be made of any material by method such as laser etching.

At step 320, the module bearing the template design then makes direct contact with an electrical element layer of a pre-treated substrate (e.g., substrate 10 depicted in FIG. 2). The electrical element layer is selected from the group consisting of a metal layer, an insulating layer, a layer of photoresist, and a combination thereof. In some embodiments, the electrical element layer is at the top of the substrate.

At step 330, a pattern is printed on the electrical element layer and the pattern is formed by a material having a photo sensitivity. For example, the material can be positive or negative photoresist.

After the pattern is printed, a plurality of electrical elements of the electrical device is created at step 340 by using a variety of methods, including but not limited to ultraviolet photolithography, etching, deep reactive ion etching, ion milling, grinding, polishing, and etc.

At step 350, a decision is made as to whether there are additional electrical elements to be defined and created. If there are additional electrical elements to be defined and created, a new electrical element layer is deposited over the previous layer and the process returns to step 310 and cycle through step 350.

One of skill in the art would understand that, in some embodiments, no additional electrical element is needed and further processing can be carried out on the same electrical layer (e.g., step 360).

One of skill in the art would understand that, for more simple devices, it is possible to create the entire device in one cycle. For more complex devices, multiple cycles of steps 310 through 350 will be needed until all features of the electrical device are completed.

Once the all features of the electrical device are completed, post-printing processing steps take place (e.g., at step 370); for example, substrate material, including any buried layer such as a BOX layer are removed to release individual devices manufactured by steps 310-360 or cycles of 310-360. At step 380, the completed individual devices are further processed (e.g., cleaned, treated and stored) before they are used.

FIG. 3B depicts a similar process comprising steps 310-a through 380-a with some variations. A module with a pre-made template design is also used to print a pattern on an electrical element layer. In these embodiments, however, the electrical element layer also has a photo sensitivity that is different from the photo sensitivity of the material used to form the printed pattern. For example, one is a negative photoresist and the other is a positive photoresist.

The processes described herein enable electrical elements having features that are as small as 0.3 μm. In some embodiments, the features are 5 μm or smaller, 4.5 μm or smaller, 4 μm or smaller, 3.5 μm or smaller, 3 μm or smaller, 2.5 μm or smaller, 2 μm or smaller, 1.8 μm or smaller, 1.5 μm or smaller, 1.2 μm or smaller, 1.0 μm or smaller, 0.8 μm or smaller, 0.6 μm or smaller, 0.4 μm or smaller, 0.2 μm or smaller, 0.1 μm or smaller, 0.05 μm or smaller, or 0.05 μm or smaller.

### Design of Arrangements of Electrical Elements: Neural Microsystems/Probes

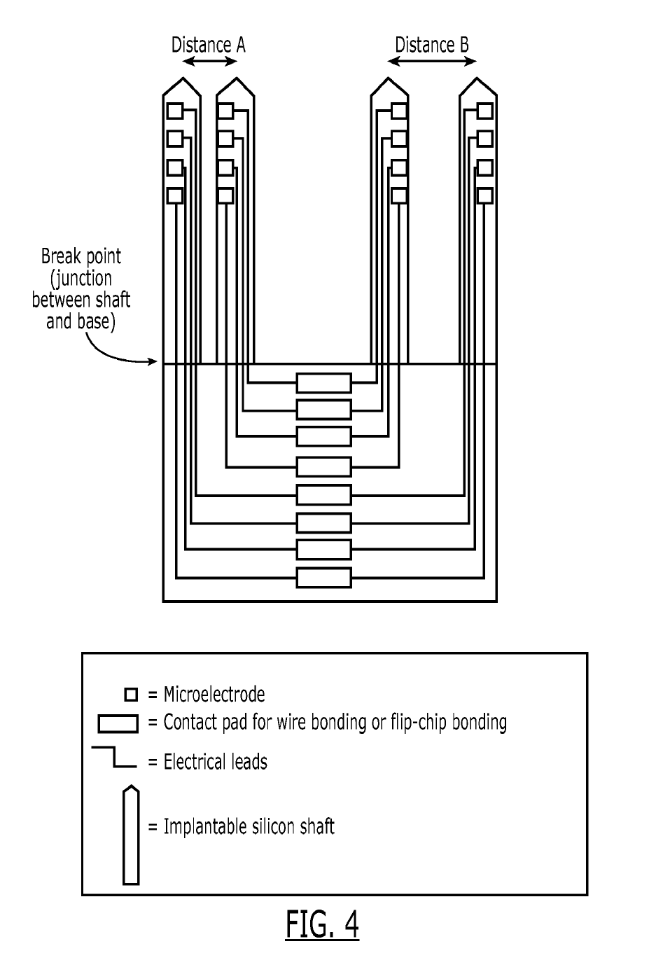

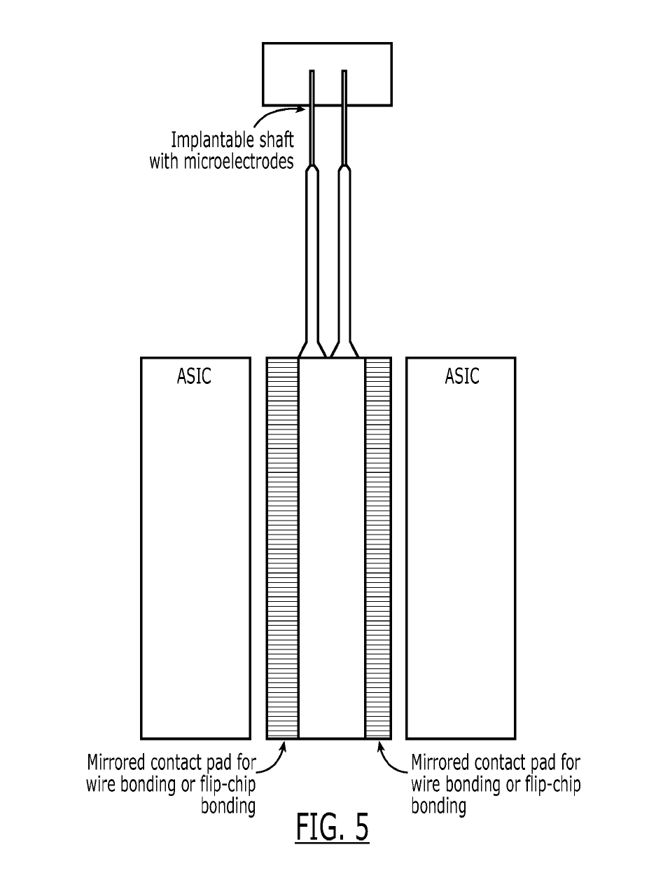

One of skill in the art will understand that the methods and processes described herein can be applied to manufacture any arrangement of electrical elements, including but not limited to an integrated circuit (“IC,” also known as a chip or microchip), or more integrated devices such as a stand-alone probe (e.g., as shown in FIGS. 4 and 5). Microsystems or probes for recording neural activities of the brain are provided here by way of example and should not in any way limit the scope of the invention.

The conventional approach to targeting multiple brain structures, or different regions of the same structure in vivo, has been to deploy multiple electrodes on multiple penetrating shafts. In this way, each shaft could target a different area. The pitfall of this approach is that the spacing between shafts on a monolithic silicon device is fixed; on the other hand the brain's anatomy raises the need for probing multiple areas at a variety of length scales. The conventional approach to resolving this issue is to manufacture a large number of different probe designs.

FIG. 4 illustrate a novel design of an exemplary probe design, which allows simultaneous targeting multiple brain structures.

In the novel approach, probes are manufactured with an excess number of implantable shafts providing greater design variability on the same monolithic device. This is achieved by connecting each wire bond (or flip-chip, or other contact method) contact pad to more than one recording electrode, where each electrode lies on a different implantable shaft.

In some embodiment, a probe comprises 2 or more implantable shafts, 3 or more implantable shafts, 4 or more implantable shafts, 5 or more implantable shafts, 6 or more implantable shafts, 7 or more implantable shafts, 8 or more implantable shafts, 9 or more implantable shafts, 10 or more implantable shafts, 12 or more implantable shafts, 15 or more implantable shafts, 18 or more implantable shafts, 20 or more implantable shafts, 25 or more implantable shafts, 30 or more implantable shafts, 35 or more implantable shafts, 40 or more implantable shafts, 50 or more implantable shafts, 60 or more implantable shafts, 70 or more implantable shafts, 80 or more implantable shafts, 90 or more implantable shafts, or 100 or more implantable shafts.

In some embodiment, a probe comprises 2 or more implantable shafts, 4 or more implantable shafts, 8 or more implantable shafts, 16 or more implantable shafts, 32 or more implantable shafts, or 64 or more implantable shafts.

In some embodiments, the electrodes being connected to the same contact pad are located on different implantable shafts. In some embodiments, more than one electrodes being connected to the same contact pad are located on the same implantable shaft.

In some embodiments, multiple electrodes are distributed on each implantable shaft. In some embodiment, an implantable shaft comprises 2 or more electrodes, 4 or more electrodes, 5 or more electrodes, 8 or more electrodes, 10 or more electrodes, 15 or more electrodes, 20 or more electrodes, 30 or more electrodes, 40 or more electrodes, 50 or more electrodes, 60 or more electrodes, 70 or more electrodes, 80 or more electrodes, 90 or more electrodes, 100 or more electrodes, 120 or more electrodes, 150 or more electrodes, 180 or more electrodes, 200 or more electrodes, 250 or more electrodes, 300 or more electrodes, 500 or more electrodes, 1,000 or more electrodes, or 2,000 or more electrodes.

In some embodiment, each implantable shaft comprises 2 or more electrodes, 4 or more electrodes, 8 or more electrodes, 16 or more electrodes, 32 or more electrodes, 64 or more electrodes, 128 or more electrodes, 256 or more electrodes, 512 or more electrodes, or 1,024 or more electrodes.

In some embodiments, the implantable shafts are configured such that they form a planar arrangement. In some embodiments, the implantable shafts are configured such that they form a three-dimensional arrangement.

In some embodiments, larger external contact pads are used to convey electrical signals off the probe to a printed circuit board or integrated circuit via wire bonding or flip-chip bonding.

In some embodiments, each probe comprise 2 or more contact pads, 4 or more contact pads, 5 or more contact pads, 8 or more contact pads, 10 or more contact pads, 15 or more contact pads, 20 or more contact pads, 30 or more contact pads, 40 or more contact pads, 50 or more contact pads, 60 or more contact pads, 70 or more contact pads, 80 or more contact pads, 90 or more contact pads, 100 or more contact pads, 120 or more contact pads, 150 or more contact pads, 180 or more contact pads, 200 or more contact pads, 250 or more contact pads, 300 or more contact pads, 500 or more contact pads, 1,000 or more contact pads, or 2,000 or more contact pads.

In some embodiment, each probe comprises 2 or more contact pads, 4 or more contact pads, 8 or more contact pads, 16 or more contact pads, 32 or more contact pads, 64 or more contact pads, 128 or more contact pads, 256 or more contact pads, 512 or more contact pads, or 1,024 or more contact pads.

In some embodiments, each contact pad is connected to two electrodes as depicted in FIG. 1. In some embodiments, the contact pad is connected to more than two electrodes, for example, three or more electrodes, four or more electrodes, five or more electrodes, six or more electrodes, seven or more electrodes, eight or more electrodes, nine or more electrodes, ten or more electrodes, 15 or more electrodes, 20 or more electrodes, 25 or more electrodes, 30 or more electrodes, 40 or more electrodes, 50 or more electrodes, 60 or more electrodes, 80 or more electrodes, 100 or more electrodes, 120 or more electrodes, 150 or more electrodes, 180 or more electrodes, 200 or more electrodes, 250 or more electrodes, 300 or more electrodes, 400 or more electrodes, 500 or more electrodes, or 600 or more electrodes.

In some embodiments, each contact pad is connected to more than two electrodes, four or more electrodes, eight or more electrodes, 16 or more electrodes, 32 or more electrodes, 64 or more electrodes, or 128 or more electrodes.

This novel approach stands in stark contrast to previous embodiments of silicon-based neural probes, which utilize one recording electrode per contact pad. In some embodiments, a user will mechanically break the undesired shafts, thereby allowing spatially precise targeting of the desired brain areas. For example, in FIG. 4, the microelectrodes on the left hand side shafts are spaced by a distance A, and the microelectrodes on the right hand side shafts are spaced by a distance B. To use the “A” spacing the user would physically break the two shafts on the right by mechanical force. The shafts would break near the specified break point. Conversely, to use the “B” spacing the user would physically break the two shafts on the left.

In some embodiments, the electrodes are arranged in an array. One of skill in the art will understand that electrodes on each shaft can be arranged in any pattern.

In some embodiments, a user will also apply a small amount of electrically insulating epoxy or other polymer to “cap” the broken segment of the silicon probe, to prevent unwanted electrical interference.

### Neural Microsystems/Probes: Connecting Additional Devices

Any devices can be connected to a probe and electrodes therein via, for example, the contact pads. For example, as depicted in FIG. 5, a probe (e.g., a silicon-based neural probe) is connected to two application-specific integrated circuits (ASIC). In some embodiments, a probe is connected to more than two ASICs; for example, 3 or more, 4 or more, 5 or more, 6 or more, 7 or more, 8 or more, 10 or more, 12 or more, 15 or more, 20 or more, 25 or more, 30 or more ASICs.

The advent of ASICs for electrophysiology has paved the way for significant improvements in the recording capabilities of implantable microsystems. Specifically, ASICs allow signals from substantially more recording electrodes to be read out in parallel. At the same time, this raises challenges for how to connect the silicon-based probe to the ASIC. The conventional approach to connecting ASICs with neural probes has been to indirectly connect them via at least one additional connector. I propose a design variant that will allow direct connection between the probe and at least two ASICs (can be two of the same, or two different ASICs).

In some embodiments, electrical contacts are “mirrored” around the midline of the probe (FIG. 5). The spacing of the contacts of the probe matches that of the ASIC, making a straightforward connection path for a wire bonder or flip-chip bonder. One ASIC is then placed next to the left-hand set of contacts, and another ASIC is placed next to the right-hand set of contacts, as depicted in FIG. 5. Any suitable ASICs can be connected with the contact pads; for example, those commercially available from Intan Technologies (Los Angeles, Calif.).

In some embodiments, a probe is connected indirectly to multiple application-specific integrated circuits.

In some embodiments, the silicon neural probe is wire-bonded to a printed circuit board (PCB 1). PCB 1 contains one or more Molex SlimStack mezannine connectors with 0.3 to 0.5 mm pitch. The connectors mate with one or more corresponding Molex SlimStack connectors on one or more PCB's containing ASICs and other electronic components for performing signal preamplification, amplification, filtering, and multiplexing; for example, those commercially available from Intan Technologies (Los Angeles, Calif.).

In some embodiments, the probes can be operated by assembling them onto a printed circuit board, and directly implanting them in the brain of a live animal (such as, a mouse, a rat, a bird, or a human) and recording the ensuing neuronal activities.

In some embodiments, the neuronal activities will be recorded continuously over a period of time, from minutes, hours, days to months.

### Neural Microsystems/Probes: Material

In some embodiments, a device provided herein such as a multi-electrode microsystem/probe comprises three classes of materials: (i) silicon (e.g., 15 to 50 microns thick) will serve as the mechanical support material; (ii) metal (e.g., including but not limited to titanium, gold, platinum) will comprise the electrodes, electrical leads, and contact sites for wire bonding or flip chip bonding; and (iii) oxide of silicon and/or silicon nitride and/or parylene C will comprise the electrical insulating layers on either side of the metal.

In some embodiments, the thickness of the device layer is less than 1000 microns, less than 800 microns, less than 700 microns, less than 600 microns, less than 500 microns, less than 300 microns, less than 200 microns, less than 150 microns, less than 100 microns, less than 75 microns, less than 50 microns, less than 45 microns, less than 40 microns, less than 35 microns, less than 30 microns, less than 25 microns, less than 20 microns, less than 18 microns, less than 15 microns, less than 12 microns, less than 10 microns, less than 8 microns, less than 6 microns, and less than 4 microns.

In some embodiment, the silicon device layer has a thickness of 300-750 microns.

In some embodiments, the substrate further comprises a buried oxide (BOX) layer having thickness from 0.01 to 2 microns.

In some embodiments, the BOX layer has a thickness of 0.01 to 0.10 microns, 0.01 to 0.20 microns, 0.01 to 0.30 microns, 0.01 to 0.40 microns, 0.01 to 0.50 microns, 0.01 to 0.60 microns, 0.01 to 0.70 microns, 0.01 to 0.80 microns, 0.01 to 0.90 microns, 0.01 to 1.0 microns, 0.01 to 1.1 microns, 0.01 to 1.2 microns, 0.01 to 1.3 microns, 0.01 to 1.4 microns, 0.01 to 1.5 microns, 0.01 to 1.6 microns, 0.01 to 1.7 microns, 0.01 to 1.8 microns, 0.01 to 1.9 microns, or 0.01 to 2.0 microns. In some embodiments, the BOX layer has a thickness of 2.0 microns or more, 2.2 microns or more, 2.5 microns or more, 2.8 microns or more, 3.0 microns or more, 3.5 microns or more, 4.0 microns or more, 5.0 microns or more. In some embodiments, the BOX layer has a thickness of 0.01 micro or less.

### Neural Microsystems/Probes: Applications

In some embodiments, probes disclosed herein are used in model laboratory organisms such as mice and rats. These animal studies have the potential to serve the academic neuroscience research community, as well as the pharmaceuticals industry in initial testing of neuropsychopharmaceuticals. The low production cost associated with this method, as well as the enhanced performance of these devices will ensure the devices can penetrate previously inaccessible markets.

In some embodiments, probes disclosed herein are used to conduct translational neuroscience research or treatment in human subjects (e.g., to study the brain-machine interfaces).

One of skill in the art will understand that probes disclosed herein can be used in any suitable diagnostic or therapeutic applications, including but not limited to those disclosed in U.S. Pat. No. 8,355,768 to Masmanidis et al. and entitled “Micromachined neural probes;” which is hereby incorporated by reference herein in its entirety.

Having described the invention in detail, it will be apparent that modifications, variations, and equivalent embodiments are possible without departing the scope of the invention defined in the appended claims. Furthermore, it should be appreciated that all examples in the present disclosure are provided as non-limiting examples.

### EXAMPLES

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein. A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

### Example 1

### Exemplary Manufacturing of a Neural Probe

FIG. 5 depicts a neural probe with two arrays of mirrored contact pads for wire bonding or flip-chip bonding. To each array of contact pads is attached an ASIC.

In this example, the neural probe depicted in FIG. 5 was manufactured according to processes and methods described herein.

Fabrication took place on a 150 mm (6 inch) square silicon-on-oxide (SOI) substrate. A stepper mask aligner with a module bearing a square or rectangular reticle design was used to repeat a square or rectangular reticle pattern across the wafer. The reticle contained patterns for multiple variants of neural probes. The silicon device layer or substrate have thickness ranging from 10-50 microns.

The process steps were carried out as follows:
(i) depositing a layer of stress-free or low stress silicon nitride (e.g., at a thickness of 0.5 to 2 microns) on the top side of a substrate;
(ii) blanket-depositing Titanium/Gold/Platinum or a subset of these metals;
(iii) using a stepper with a first module to stamp selected areas with photoresist, exposing the areas to UV radiation, and using dry etching to selectively removed unstamped areas, resulting in definition of electrodes, electrical leads, and contact pads;
(iv) depositing low stress or stress-free silicon nitride (e.g., at a thickness of 0.5 to 2 microns) on the top side;
(v) using a stepper with a second module to stamp selected areas with photoresist, exposing the areas to UV radiation, and using dry etching to selectively remove the upper layer of nitride, thereby exposing the metal over the contact pads and electrodes;
(vi) using a stepper with a second module to stamp selected areas with photoresist, exposing the areas to UV radiation, and using dry etching to etch through the remaining silicon nitride and silicon device layer and the reveal portions of the BOX layer, resulting in the definition of a sharp implantable shafts;
(vii) mounting the SOI wafer upside down on a wafer carrier using Crystalbond 555 wax;
(viii) optionally, thinning down the back side of the SOI wafer with a wafer grinding or polishing tool, until a thickness of 50-150 microns is reached;
(ix) etching the remaining silicon handle layer with deep reactive ion etching (DRIE) until the BOX layer is reached;
(x) using DRIE or other dry etching to remove the remaining BOX layer;
(xi) releasing the devices by pouring or immersing the wafer carrier in hot water (60-100° C.) and fishing out individual devices with tweezers or a vacuum wand;
(xii) cleaning individual devices in deionized water, acetone, and ethanol or a subset of these solvents; and
(xiii) drying the devices and storing them for use.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

Many variations and alternative elements have been disclosed in embodiments of the present invention. Still further variations and alternate elements will be apparent to one of skill in the art. Various embodiments of the invention can specifically include or exclude any of these variations or elements.

In some embodiments, the numbers expressing quantities of ingredients, properties such as molecular weight, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

1. A process for manufacturing a device comprising a plurality of electrical elements, comprising:
applying a first module in direct contact with a first electrical element layer of a pre-treated substrate, wherein the first electrical element layer is selected from the group consisting of a metal layer, an insulating layer, a semiconductor layer, a layer of photoresist, and a combination thereof, and wherein the first module comprises, on a surface that forms direct contact with the first electrical element layer, a first template design; and
printing a first pattern on the first electrical element layer, wherein the first pattern is formed by a first material having a photo sensitivity.

2. The process of claim 1, wherein the first electrical element layer has a first photo sensitivity that is different from the photo sensitivity of the first material.

3. The process of claim 1, wherein the first material is a photoresist and the first pattern is exposed to ultraviolet radiation.

4. The process of claim 1, further comprising:
creating a first plurality of electrical elements of the device using one or more methods selected from the group consisting of ultraviolet photolithography, etching, deep reactive ion etching, ion milling, grinding, polishing, and combinations thereof.

5. The process of claim 1, further comprising:
creating the first template design on the first module, wherein the first template design corresponding to an arrangement of a first plurality of electrical elements of the device, and wherein at least one electrical element of the first plurality is selected from the group consisting of an electrode, an electrical lead, an insulator, a contact pad, a semiconductor, and a combination thereof.

6. The process of claim 1, further comprising:
depositing a second electrical element layer above the first electrical element layer, wherein the second electrical element layer is selected from the group consisting of a metal layer, an insulating layer, a semiconductor layer, a layer of photoresist, and a combination thereof.

7. The process of claim 6, wherein the second electrical element layer is different from the first electrical element layer.

8. The process of claim 6, further comprising:
applying a second module in direct contact with the second electrical element layer, wherein the second module comprises, on a surface that forms direct contact with the second electrical element layer, a second template design; and
printing a second pattern on the second electrical element layer, wherein the second pattern is formed by a second material having a photo sensitivity.

9. The process of claim 8, wherein the second electrical element layer has a second photo sensitivity that is different from the photo sensitivity of the material forming the second pattern.

10. The process of claim 8, wherein the second material is a photoresist and the second pattern is exposed to ultraviolet radiation.

11. (canceled)

12. The process of claim 8, further comprising:
creating the second template design on the second module, wherein the second template design corresponding to an arrangement of a second plurality of electrical elements of the device, and wherein at least one electrical element of the second plurality is selected from the group consisting of an electrode, an electrical lead, an insulator, a contact pad, a semiconductor, and a combination thereof.

13. The process of claim 6, further comprising:
depositing a third electrical element layer above the second electrical element layer, wherein the third electrical element layer is selected from the group consisting of a metal layer, an insulating layer, a semiconductor layer, a layer of photoresist, and a combination thereof.

14. The process of claim 13, wherein the third electrical element layer is different from the second electrical element layer.

15. The process of claim 13, further comprising:
applying a third module in direct contact with the third electrical element layer, wherein the third module comprises, on a surface that forms direct contact with the third electrical element layer, a third template design; and
printing a third pattern on the third electrical element layer, wherein the third pattern is formed by a third material having a photo sensitivity.

16. The process of claim 15, wherein the third electrical element layer has a third photo sensitivity that is different from the photo sensitivity of the third material.

17. The process of claim 15, wherein the third material is a photoresist and the third pattern is exposed to ultraviolet radiation.

18. (canceled)

19. The process of claim 15, further comprising:
creating the third template design on the third module, wherein the third template design corresponding to an arrangement of a third plurality of electrical elements of the device, and wherein at least one electrical element of the third plurality is selected from the group consisting of an electrode, an electrical lead, an insulator, a contact pad, a semiconductor, and a combination thereof.

20. The process of claim 1, wherein the pre-treated substrate further comprises:
a base material selected from the group consisting of silicon, quartz, silicon oxide, sapphire, gallium arsenide, magnesium oxide, zinc oxide, a buried oxide (BOX) layer, and silicon carbide.

21-24. (canceled)

25. A device comprising a plurality of electrical elements manufactured according to the process of claim 1.

26. (canceled)

27. A neural probe, comprising:
a first implantable shaft;
a first plurality of electrodes disposed on the first implantable shaft;
a second implantable shaft;
a second plurality of electrodes disposed on the second implantable shaft;
a base to which the first implantable shaft and the second implantable shaft are attached and separated by a distance; and
at least one contact pad on the base to which one electrode from the first plurality of electrodes and one electrode from the second plurality of electrodes are connected.

28-36. (canceled)
